(19) United States
(12) Patent Application Publication
(10) Pub. No.: US 2010/0234735 A1
(43) Pub. Date: Sep. 16, 2010

(54) ULTRASOUND PROBE AND AN ULTRASONIC DIAGNOSIS APPARATUS

(75) Inventor: Hideki KOSAKU, Nasushiobara-shi (JP)

Correspondence Address: OBLON, SPIVAK, MCCLELLAND MAIER & NEUSTADT, L.L.P., 1940 DUKE STREET, ALEXANDRIA, VA, 22314, US

(73) Assignees: KABUSHIKI KAISHA TOSHIBA, Tokyo (JP); TOSHIBA MEDICAL SYSTEMS CORPORATION, Otawara-shi (JP)

(21) Appl. No.: 12/719,288

(22) Filed: Mar. 8, 2010

(30) Foreign Application Priority Data
Mar. 11, 2009 (JP) 2009-057382

(51) Int. Cl. A61B 8/14; H01L 41/053

(52) U.S. Cl. 600/459; 310/348

(57) ABSTRACT

A tube has a hollow portion and at its tip end, a recess with an open window. A rotating member is held in the recess and is rotated about a central axis. A driving cable transmits a rotating force to the rotating member (or a feeding cable supplies an electric power to a drive part for rotating the rotating member). A piezoelectric vibrator is supported by the rotating member so as to swing its ultrasonic-wave transmitting/receiving surface about a swing axis that is orthogonal to the central axis. A permanent magnet is provided on one of the rotating member and the piezoelectric vibrator. An electromagnet is provided on the other of the rotating member and the piezoelectric vibrator so as to be opposed to the permanent magnet. And a power supply cable supplies, to the electromagnet, a current for swinging the piezoelectric vibrator.

### BACKGROUND OF THE INVENTION

1. Field of the Invention

The present invention relates to an ultrasound probe and an ultrasonic diagnosis apparatus.

The present invention particularly relates to an ultrasound probe and an ultrasonic diagnosis apparatus in which the direction of a piezoelectric vibrator may be turned.

2. Description of the Related Art

Examples of ultrasound probes in which the direction of a piezoelectric vibrator may be turned include a technology (transesophageal echocardiography (TEE) ultrasound probe) in which the piezoelectric vibrator is rotated about the central axis of the ultrasonic image in the same way as in a transesophageal probe, etc. to obtain a three-dimensional image. There is also a technology (mechanical 4D probe) in which the piezoelectric vibrator is rotated in the orthogonal direction relative to the direction of scanning of the image to obtain a three-dimensional image. Additionally provided are: a swing mechanism that swings the piezoelectric vibrator, which is disposed in a receiving part provided in the tip end of a tube, about an axis parallel to an ultrasonic-wave transmitting/receiving surface; and a rotation mechanism that rotates the piezoelectric vibrator about an axis vertical to the ultrasonic-wave transmitting/receiving surface.

Moreover, in one example of the prior art (Unexamined Patent Application Publication No. H8-84732), a rotation mechanism comprises: a seat provided with a projected spherical surface; a receiving seat in which a recessed spherical surface is formed with a curvature that may be engaged with this projected spherical surface and supports the seat to allow the seat to swing by engaging the projected spherical surface with the recessed spherical surface, and a swing wire that facilitates the swinging of the abovementioned seat.

However, in the abovementioned TEE ultrasound probe, the rotational speed of the piezoelectric vibrator is low and it therefore takes a long time to obtain data, which makes it difficult to construct a three-dimensional image with a sufficient image quality. Furthermore, in the abovementioned mechanical 4D probe, the image quality is not good in the direction orthogonal to the scanning direction. Moreover, in the technology related to the patent literature in which the swing and rotation of the piezoelectric vibrator have been combined, the piezoelectric vibrator is swung by pulling the wire in and out, thereby causing operational delays and errors due to the friction and slack of the wire, leading to the problem that the image quality of the three-dimensional image is reduced. What is desired is a probe that includes the functions of both the abovementioned TEE ultrasound probe and mechanical 4D probe and is capable of obtaining three-dimensional images in real time.

### SUMMARY OF THE INVENTION

This invention is intended to overcome the above problems by providing an ultrasonic probe and an ultrasonic diagnosis apparatus allowing high quality three dimensional image to be obtained in real time.

A first aspect of the present invention is an ultrasound probe comprising: a tube having a hollow portion that is formed in a long-axis shape and is extended in the direction of said long axis as well as, at a tip end thereof, a recess with an open window allowing ultrasonic waves to pass; a rotating member that is held in said recess and is rotated about a central axis oriented in the direction from the inside of said recess to said window; a driving cable that passes through said hollow portion and is used for transmitting a rotating force to said rotating member, or a feeding cable that supplies an electric power to a drive part for rotating said rotating member; a piezoelectric vibrator that has an ultrasonic-wave transmitting/receiving surface and is supported by said rotating member so as to swing said ultrasonic-wave transmitting/receiving surface about a swing axis that is orthogonal to said central axis; a permanent magnet that is provided on one of said rotating member and said piezoelectric vibrator; an electromagnet that is provided on the other of said rotating member and said piezoelectric vibrator so as to be opposed to said permanent magnet; and a power supply cable that passes through said hollow portion and supplies, to said electromagnet, a current for swinging said piezoelectric vibrator.

According to the first aspect of this invention, a piezoelectric vibrator having a surface of transmitting/receiving ultrasound is supported on a rotating member, allowing high quality three dimensional image to be obtained. Further, since current is supplied to an electromagnet to swing the piezoelectric vibrator around a swinging axis, delay or error of an behavior of the piezoelectric vibrator is prevented, allowing three dimensional image to be obtained in real time.

A second aspect of the present invention is the ultrasound probe according to the first aspect of this invention, wherein: said rotating member has a cylindrical shape with a cylindrical wall and a bottom and is shaped substantially coaxially with said recess; said piezoelectric vibrator is held within said rotating member; said swing axis is installed between opposing parts of said cylindrical wall; said ultrasonic-wave transmitting/receiving surface is formed toward said window relative to said swing axis; one of said permanent magnet and said electromagnet is provided toward said bottom relative to said swing axis; and the other of said permanent magnet and said electromagnet is provided on both sides of said one magnet in said bottom.

A third aspect of the present invention is the ultrasound probe according to the first aspect of this invention, wherein: said rotating member is cylindrically shaped; an input gear is integrally provided at the bottom of said rotating member; a rotating axis of a motor is connected to the base end of said driving cable; a worm gear is connected to the tip end of said driving cable; and said worm gear is engaged with said input gear via a speed reducing gear.

A fourth aspect of the present invention is the ultrasound probe according to the first aspect of this invention, comprising: a swing-angle detecting part that detects the swing angle of said piezoelectric vibrator relative to said rotating member; and a swing-angle controlling part that controls said current supplied to said electromagnet upon receiving a detection signal from said swing-angle detecting part.

A fifth aspect of the present invention is the ultrasound probe according to the fourth aspect of this invention, wherein: a magnetic body is provided to one of said rotating member and said piezoelectric vibrator; and said swing-angle detecting part is provided to the other of said rotating member and said piezoelectric vibrator and detects the magnetic force of said magnetic body.

A sixth aspect of the present invention is an ultrasonic diagnosis apparatus with an ultrasound probe, the ultrasound probe comprising: a tube having a hollow portion that is formed in a long-axis shape and is extended in the direction of said long axis as well as, at a tip end thereof, a recess with an open window allowing ultrasonic waves to pass; a rotating member that is held in said recess and is rotated about a central axis oriented in the direction from the inside of said recess to said window; a driving cable that passes through said hollow portion and is used for transmitting a rotating force to said rotating member, or a feeding cable that supplies an electric power to a drive part for rotating said rotating member; a piezoelectric vibrator that has an ultrasonic-wave transmitting/receiving surface and is supported by said rotating member so as to swing said ultrasonic-wave transmitting/receiving surface about a swing axis that is orthogonal to said central axis; a permanent magnet that is provided on one of said rotating member and said piezoelectric vibrator; an electromagnet that is provided on the other of said rotating member and said piezoelectric vibrator so as to be opposed to said permanent magnet; and a power supply cable that passes through said hollow portion and supplies, to said electromagnet, a current for swinging said piezoelectric vibrator.

A seventh aspect of the present invention is the ultrasonic diagnosis apparatus according to the sixth aspect of this invention, wherein: said rotating member has a cylindrical shape with a cylindrical wall and a bottom and is shaped substantially coaxially with said recess; said piezoelectric vibrator is held within said rotating member; said swing axis is installed between opposing parts of said cylindrical wall; said ultrasonic-wave transmitting/receiving surface is formed toward said window relative to said swing axis; one of said permanent magnet and said electromagnet is provided toward said bottom relative to said swing axis; and the other of said permanent magnet and said electromagnet is provided on both sides of said one magnet in said bottom.

An eighth aspect of the present invention is the ultrasound probe according to the sixth aspect of this invention, wherein: said rotating member is cylindrically shaped; an input gear is integrally provided at the bottom of said rotating member; a rotating axis of a motor is connected to the base end of said driving cable; a worm gear is connected to the tip end of said driving cable; and said worm gear is engaged with said input gear via a speed reducing gear.

A ninth aspect of the present invention is the ultrasound probe according to the sixth aspect of this invention, comprising: a swing-angle detecting part that detects the swing angle of said piezoelectric vibrator relative to said rotating member; and a swing-angle controlling part that controls said current supplied to said electromagnet upon receiving a detection signal from said swing-angle detecting part.

A tenth aspect of the present invention is the ultrasound probe according to the ninth aspect of this invention, wherein: a magnetic body is provided to one of said rotating member and said piezoelectric vibrator; and said swing-angle detecting part is provided to the other of said rotating member and said piezoelectric vibrator and detects the magnetic force of said magnetic body.

### BRIEF DESCRIPTION OF THE DRAWINGS

FIG. 1 is a cross-sectional view of an ultrasound probe showing the inside of a tube along the long axis in connection with the embodiment of the present invention.

FIG. 2 is a partial perspective view of an ultrasound probe.

FIG. 3 is a cross-sectional view along the line III-III of FIG. 1.

FIG. 4 is a functional block diagram of an ultrasound probe.

FIG. 5 is a conceptual diagram showing each operation of the rotation and swinging of the piezoelectric vibrator.

FIG. 6A-C show each magnet body arranged in a fan shape and a piezoelectric vibrator swung to each position.

FIG. 7A-H show a permanent magnet and an electromagnet that is swung to each position by being supplied with an alternating current.

FIG. 8A-C shows a piezoelectric vibrator that is held in a rotating member and is swung to each position.

FIG. 9 is a block diagram showing a structure of an ultrasonic diagnosis apparatus in connection with the embodiment of the present invention.

### DETAILED DESCRIPTION OF THE EMBODIMENTS

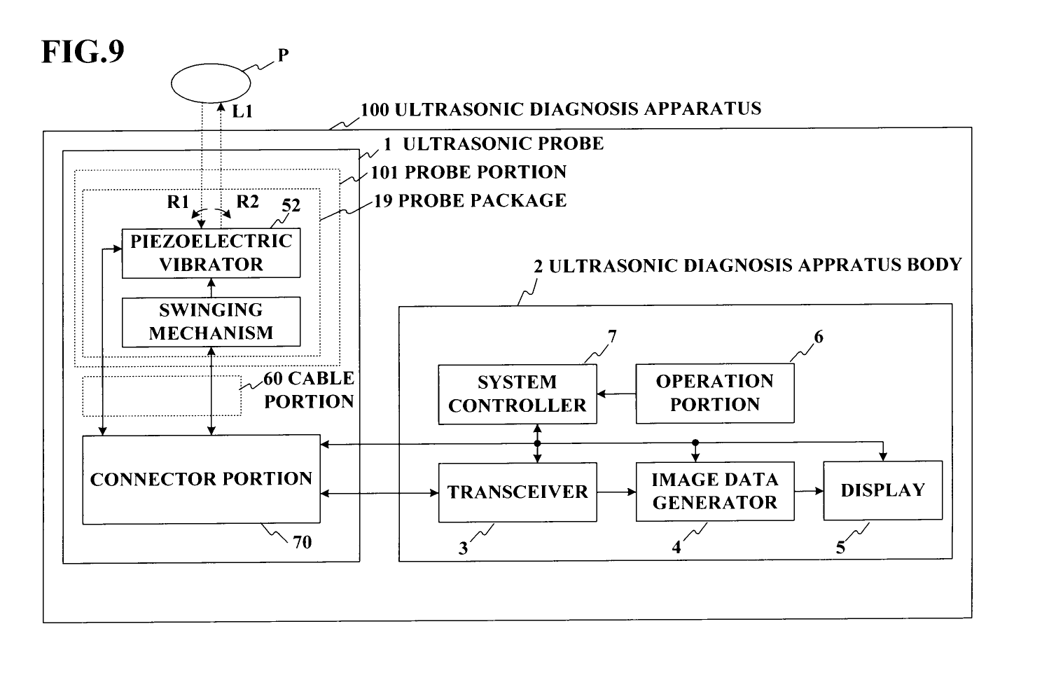

FIG. 9 is a block diagram showing a structure of an ultrasonic diagnosis apparatus in connection with the embodiment of the present invention. This ultrasonic diagnosis apparatus 100 is provided with an ultrasonic probe 1 for transmitting/receiving ultrasound to/from a subject P and an ultrasonic diagnosis apparatus body 2 for controlling this ultrasonic probe 1.

The ultrasonic probe 1 is provided with a probe portion 101 for transmitting/receiving ultrasound; a cable portion 60 connected to this probe portion at its end; and a connector portion 70 connected at the other end of this cable portion 60 for transmitting/receiving signal to/from the ultrasonic diagnosis apparatus body 2.

The probe portion 101 is provided with an electrically safe probe package 19 which is structurally superior in terms of weather resistance and environment resistance. This probe package is made of a resin material to form an outer shell of the probe portion 101. A recess 24 inside the probe package 19 is provided with an piezoelectric vibrator 52 for transmitting/receiving ultrasound and a swinging mechanism (described later) for swinging this piezoelectric vibrator 52 in a direction of arrows R1 and R2. It should be noted that an acoustic window (hereinafter, simply referred to as “window”) 23, which is a portion of the probe package 19 for transmitting/receiving ultrasound to/from a subject P, is made of a material superior for propagating ultrasound. Further, an acoustic medium superior for propagating ultrasound is enclosed between the window 23 and the piezoelectric vibrator 52.

The ultrasonic diagnosis apparatus body 2 comprises a transceiver 3 for transmitting ultrasonic driving signal and receiving ultrasonic receiving signal; and an image data generator 4 for generating two-dimensional image data based on the receiving signal from the transceiver 3 (e.g. B-mode image data representing a cross section of the subject P; and a Doppler image data representing blood flow) and for generating three-dimensional image data from the two-dimensional image data generated from several swinging angles by swinging of the piezoelectric vibrator 52 at the probe portion 101 of the ultrasonic probe 1.

Further, the ultrasonic diagnosis apparatus body 2 is provided with a display 5 for displaying two-dimensional data and/or three dimensional data generated by the image data generator 4; an operation portion 6 for input of various kinds of command signal; a system controller 7 for controlling the swinging mechanism in the probe portion 101 of the ultrasonic probe 1, the transceiver 3, the image data generator 4 and the display 5.

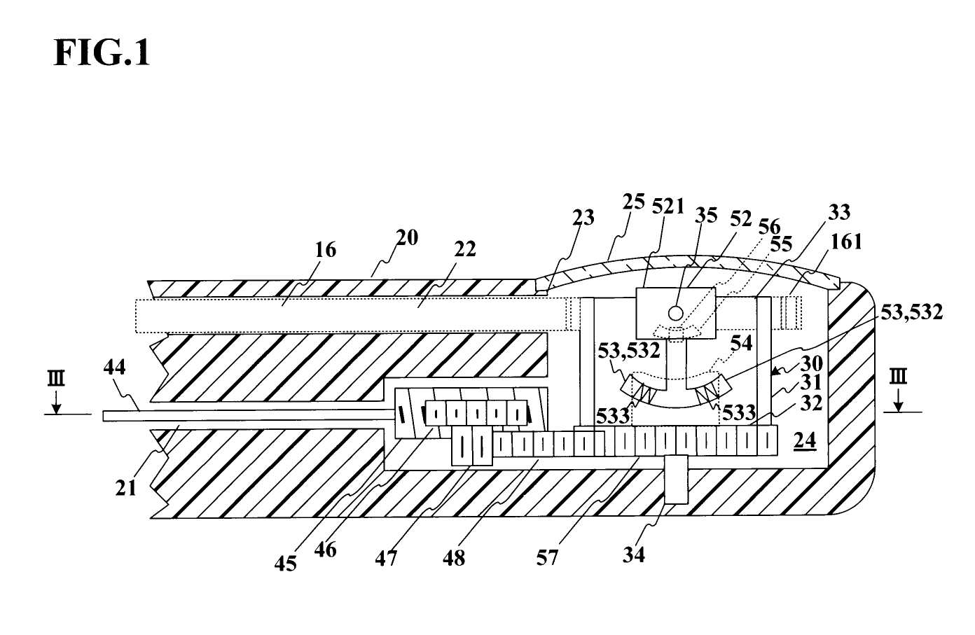

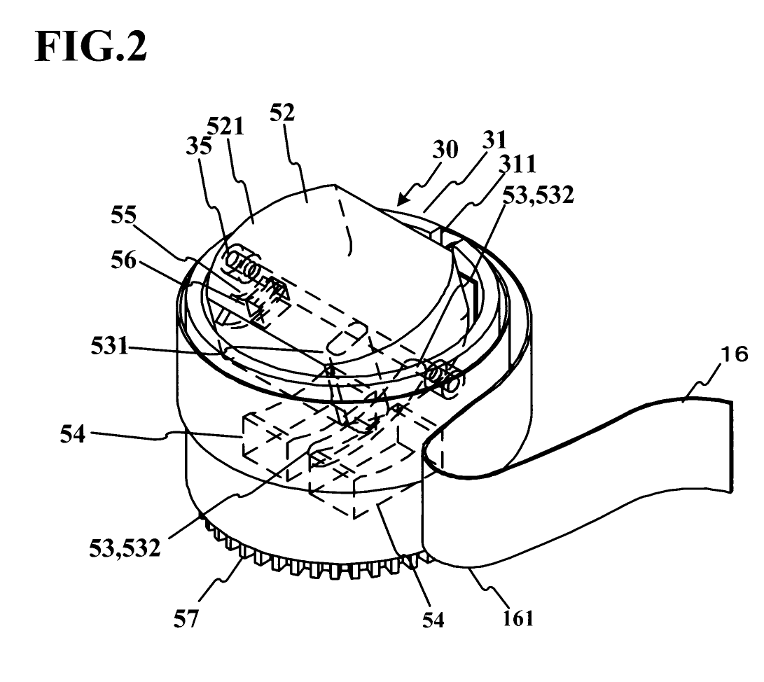

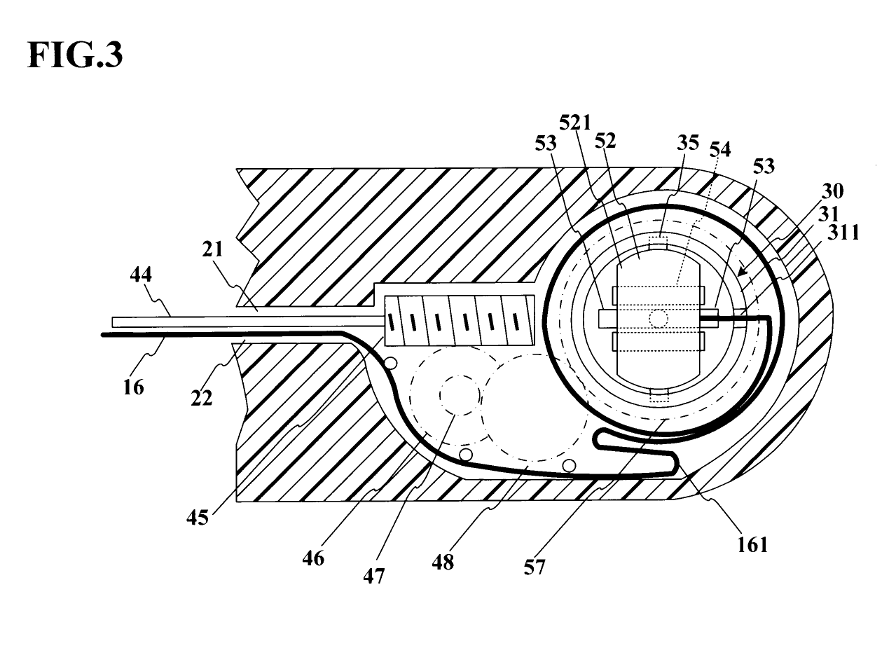

The configuration of the ultrasound probe according to one embodiment of the present invention will be described with reference to FIGS. 1-3. FIG. 1 is a cross-sectional view of an ultrasound probe showing the inside of a tube along the long axis. FIG. 2 is a partial perspective view of an ultrasound probe that is incorporated within the tip of a tube, shown with the tube omitted. FIG. 3 is a cross-sectional view along the line III-III of FIG. 1.

The ultrasound probe mainly comprises a tube 20, the piezoelectric vibrator 52, a rotation mechanism for rotating the piezoelectric vibrator 52, and the swing mechanism for swinging the piezoelectric vibrator 52.

The tube 20 is formed in a long-axis shape. In this embodiment, the tube 20 has flexibility and, in a transesophageal probe, includes a tip end thereof as well as a curve and an intermediate guide tube that continues to the tip end. The tip end of the tube 20 is provided with the recess 24. The window 23 in the recess 24 is open to the side surface of the tip end of the tube 20. It is noted that an operating part (not shown) is formed in the intermediate portion of the tube 20 and the base end of the tube 20 is connected to a system part (not shown). The details of the operating part and the system part will be described below.

The piezoelectric vibrator 52 has an ultrasonic-wave transmitting/receiving surface 521. The ultrasonic-wave transmitting/receiving surface 521 comprises an array of many piezoelectric elements formed from piezoelectric bodies such as piezoelectric ceramics. A cable 16 is provided to perform transmissions and receptions with each piezoelectric element of the ultrasonic-wave transmitting/receiving surface 521. The cable 16 is a flexible printed circuit (FPC) and includes a power supply cable for supplying an alternating current to an electromagnet 53 and a signal cable for transmitting a detection signal from a swing-angle detecting part 56 to a swing-angle controlling part (not shown). The cable 16 extends from the tip end of the tube 20 to the base end of the tube 20. It is noted that “the power supply cable for supplying the current to the electromagnet 53” includes a power supply cable that supplies an alternating current and a power supply cable that supplies a direct current for generating an alternating current.

The rotation mechanism for rotating the piezoelectric vibrator 52 comprises a rotating member 30 and a driving cable 44 for transmitting a rotating force to the rotating member 30, etc. The rotating member 30 is held in the recess 24, has a cylindrical shape with a cylindrical wall 31 and a bottom 32, and is shaped approximately coaxially with the recess 24. The rotating member 30 is supported to allow it to rotate about a central axis oriented in the direction from the inside of the recess 24 to the window 23. An input gear 57 is integrally provided at the bottom 32 of the rotating member 30. FIG. 1 shows the rotating member 30 with the upper surface of the input gear 57 shown as the bottom 32. It is noted that the rotating member 30 and the input gear 57 may be formed separately. The axis part 34 is projected from the input gear 57 along the central axis. A bearing (not shown) for supporting the axis part 34 is provided at the bottom of the recess 24. It is noted that the recess 24 is filled with ultrasound propagation medium liquid (not shown) and the window 23 is sealed with a cover 25.

The driving cable 44 extends from the tip end of the tube 20 through a hollow portion 21 to the intermediate portion of the tube 20.

The base end of the driving cable 44 is connected to the rotating axis of a motor (not shown) provided in the intermediate portion of tube 20. In addition, a hollow portion 22 is provided parallel to the hollow portion 21. The cable 16 extends from the tip end of the tube 20 through the hollow portion 22 of the tube 20 to the base end of the tube 20. It is noted that FIG. 3 shows the hollow portions 21 and 22 as one hollow portion for convenience of explanation.

A worm gear 45 is connected to the tip end of the driving cable 44. The worm gear 45 is engaged with the input gear 57 via speed reducing gears 46, 47, and 48. A motor 43 is supplied with electric power from a motor-power supplying part 15. The motor-power supplying part 15 converts a voltage supplied from a power source 11 into a voltage for driving the motor. FIG. 2 omits the worm gear 45 and the speed reducing gears 46, 47, and 48.

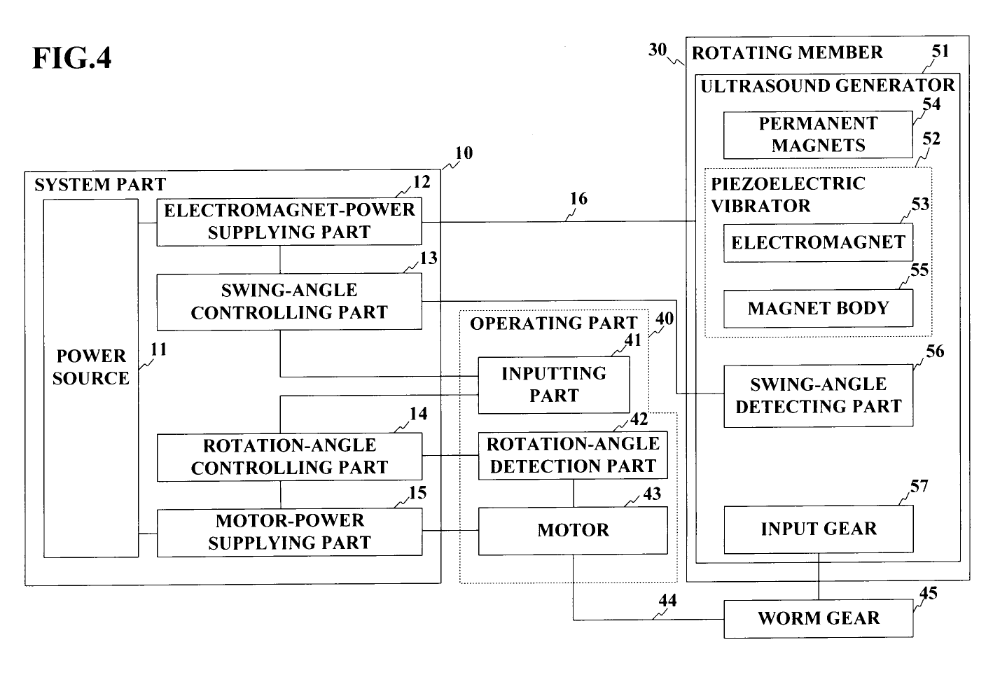

The configuration for controlling the abovementioned rotation mechanism will be described with reference to FIG. 4. FIG. 4 is a block diagram of an ultrasound probe. It is noted that the system part 10 is provided toward the base end of the tube 20. The system part 10 comprises a swing-angle controlling part 13 that controls the alternating current supplied to the electromagnet 53 upon receiving a detection signal from a swing-angle detecting part 56, and a rotation-angle controlling part 14 that controls the current supplied to the motor 43 upon receiving a detection signal from a rotation-angle detection part 42. In addition, an operating part 40 is provided in the intermediate portion of the tube 20. The operating part 40 comprises an inputting part 41, the rotation-angle detection part 42 for detecting the swing angle of the piezoelectric vibrator 52 relative to the rotating member 30, and the motor 43. The swing-angle controlling part 13 adjusts the swing angle of the piezoelectric vibrator 52 upon receiving an instruction from the inputting part 41, and the rotation-angle controlling part 14 adjusts the rotation angle of the rotating member 30 upon receiving an instruction from the inputting part 41.

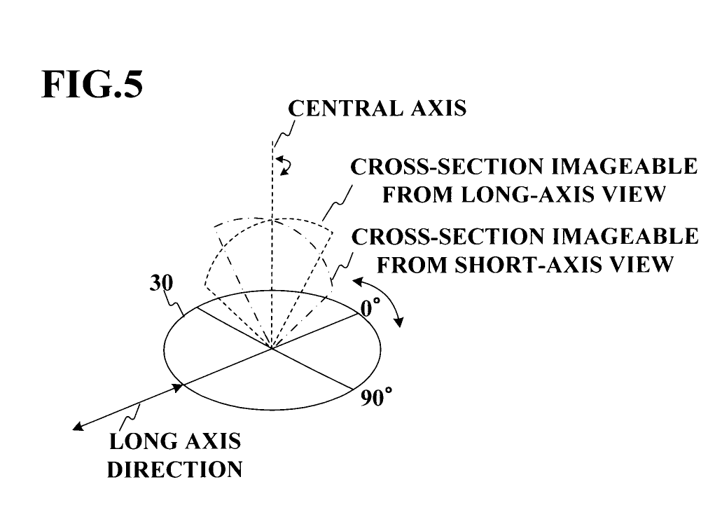

The operations of the rotating member 30 will be described with reference to FIG. 5. FIG. 5 is a conceptual diagram showing each operation of the rotation and swinging of the piezoelectric vibrator.

The motor 43 is supplied with electric power from the motor-power supplying part 15. The motor-power supplying part 15 converts a voltage supplied from the power source 11 into a voltage for driving the motor. The rotating force of the motor 43 is transmitted to the driving cable to rotate the worm gear 45. The input gear is thus rotated via the speed reducing gears 46, 47, and 48 to rotate the rotating member 30 integrally with the input gear.

The number of rotations and the rotational direction of the motor 43 correspond to the rotation angle of the rotating member 30.

The rotation-angle detection part 42 measures the number of rotations of the motor 43 and detects the rotation angle of the rotating member 30 based on the measurement result. The rotation-angle controlling part 14 controls the motor-power supplying part 15 based on the rotation angle of the rotating member 30. As the number of rotations of the motor 43 corresponds to the rotation angle of the rotating member 30, the rotating member 30 may be rotated with accuracy and high precision. FIG. 5 shows an imageable cross-section that provides long-axis and short-axis views according to the rotation angle of the rotating member 30. For example, when the rotation angle of the rotating member 30 is 0°, the imageable cross-section is to be a long-axis view, and when the rotation angle is 90°, the imageable cross section is to be a short-axis view.

The swing mechanism for swinging the piezoelectric vibrator 52 will be described with reference to FIGS. 1 to 4. The swing mechanism comprises the piezoelectric vibrator 52, the electromagnet 53, a permanent magnet 54, an electromagnet-power supplying part 12, the cable 16, and other components.

The piezoelectric vibrator 52 is supported by being held within the rotating member 30. The piezoelectric vibrator 52 having the ultrasonic-wave transmitting/receiving surface 521 is supported by the rotating member 30, thereby enabling a three-dimensional image having a high image quality to be obtained. A swing axis 35 is installed between opposing cylindrical walls 31. The ultrasonic-wave transmitting/receiving surface 521 is supported by the rotating member 30 so as to swing it about the swing axis 35 that is orthogonal to the central axis of the rotating member 30. In the piezoelectric vibrator 52 held within the rotating member 30, the ultrasonic-wave transmitting/receiving surface 521 is formed toward the window 23 relative to the swing axis 35. The ultrasonic-wave transmitting/receiving surface 521 is located at a position that is slightly projected in the direction from the position of the opening 33 of the cylindrical rotating member 30 to the window 23. It is noted that the ultrasonic-wave transmitting/receiving surface 521 may be located at a position that is recessed in the direction from the opening 33 of the rotating member 30 to the bottom 32, or may be located at a position aligned with the opening 33.

A rod 531 is integrally extended in the direction from the backside of the piezoelectric vibrator 52 (a surface opposite to the ultrasonic-wave transmitting/receiving surface 521) to the bottom 32.

The tip end of the rod 531 has 2 cores (magnetic cores) 532 that are extended in opposite directions to each other. Each core 532 is formed in an arc shape centered on the swing axis 35.

The electromagnet 53 is provided toward the bottom 32 relative to the swing axis 35. The ultrasonic-wave transmitting/receiving surface 521 thereby formed toward the window 23 relative to the swing axis 35 is swung in the direction opposite to the moving direction of the electromagnet 53.

The electromagnet 53 comprises 2 cores 532 and coils 533 wound around each core 532. In the bottom 32, the permanent magnets 54 are provided to both sides of the electromagnet 53. The rotating member 30 is held within the recess 24 and the piezoelectric vibrator 52, electromagnet 53, and permanent magnets 54 are held within the rotating member 30, thus enabling the rotation mechanism and swing mechanism to be downsized.

It is noted that the electromagnet 53 may be provided toward the rotating member 30 and the permanent magnet 54 may be provided toward the piezoelectric vibrator 52. In this case, for example, the permanent magnet 54 is provided at the tip end of the rod 531 and the electromagnets 53 are provided at the bottom 32 of the rotating member 30 and arranged on both sides of the permanent magnet 54.

The cable 16 is provided to supply, to the electromagnet 53, an alternating current for swinging the piezoelectric vibrator 52. The base end of the cable 16 is connected to an electromagnet-power supplying part 12 within the operating part 40. The tip end 161 of the cable 16 is passed through the hollow portion 22, extended to the tip end of the tube 20, pulled into the rotating member 30 through a slit 311 of the cylindrical wall 31, and connected to the electromagnet 53. The tip end 161 of the cable 16 is formed with a margin so as to follow the rotating member 30, which rotates in a forward or reverse direction. FIGS. 2 and 3 show the cable 16 with a margin having the tip end 161 bent into an “S” shape and that is about one rotation of the rotating member 30. The electromagnet 53 is supplied with electric power from the electromagnet-power supplying part 12. The electromagnet-power supplying part 12 converts a voltage supplied from the power source 11 into a voltage for driving the electromagnet.

Supplying an alternating current to the electromagnet 53 changes the polarity of the electromagnet 53 and the coil 533 is thereby attracted to and repulsed from the permanent magnet 54, thus allowing the piezoelectric vibrator 52 to be swung about the swing axis 35 and making it possible to obtain, in real time, a three-dimensional image having a high image quality without operational delay or error of the piezoelectric vibrator 52. Furthermore, it is possible to increase the durability to prevent wear-out of the cable, etc. by not using a configuration in which the piezoelectric vibrator 52 is swung by pulling in the cable.

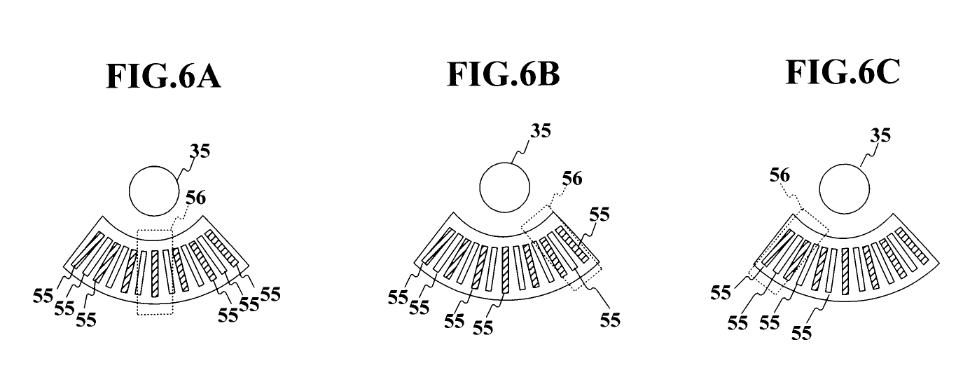

Next, the configuration for controlling the above-mentioned swing mechanism will be described with reference to FIG. 4 and FIG. 6A-C. FIG. 6A-C show each magnet body 55 arranged in a fan shape and a piezoelectric vibrator 52 swung to each position.

The configuration for controlling the swing mechanism comprises a magnetic body 55, the swing-angle detecting part 56, and the swing-angle controlling part 13.

A plurality of the magnetic bodies 55 is arranged in a fan shape about the swing axis 35 on the inner wall of the rotating member 30.

Opposite to the magnetic bodies 55, the swing-angle detecting part 56 is provided on the peripheral wall of the piezoelectric vibrator 52 to detect the swing angle of the piezoelectric vibrator 52 relative to the rotating member 30. The detected part, which is a magnetic body 55, may be provided on one of the opposing surfaces of the rotating member 30 and the piezoelectric vibrator 52, and the swing-angle detecting part 56 that is capable of detecting the swing angle relative to the rotating member 30 by detecting the detected part on the other surface may be provided, thus enabling the swing angle of the piezoelectric vibrator 52 to be detected relatively easily. The magnetic bodies 55 opposite to the peripheral wall of the piezoelectric vibrator 52 are arranged so that the N-poles and S-poles are arranged in alternation. Magnetic bodies 55 that are N-poles are indicated with hatching in FIG. 6A-C, while magnetic bodies 55 that are S-poles are shown without hatching in FIG. 6A-C. Furthermore, the swing-angle detecting part 56 is shown with dashed lines in FIG. 6A-C.

The system part 10 is provided with the electromagnet-power supplying part 12 and the swing-angle controlling part 13. The swing-angle controlling part 13 controls the alternating current supplied from the electromagnet-power supplying part 12 to the electromagnet 53 upon receiving a detection signal from the swing-angle detecting part 56. The swing-angle detecting part 56 outputs a detection signal when an N-pole magnetic body 55 and an S-pole magnetic body 55 are both detected. The swing-angle controlling part 13 counts the detection signals and when a predetermined number of detection signals are counted, it switches the direction of the current supplied to the electromagnet 53.

When the swing-angle detecting part 56 is located at a position as shown in FIG. 6A, the swing-angle detecting part 56 moves in the clockwise or counterclockwise direction relative to the magnetic body 55, and when the swing-angle controlling part 13 counts 1 detection signal, it switches the direction of the current, and additionally, when the swing-angle controlling part 13 counts 5 detection signals (6 detection signals in total), it switches the direction of the current. FIG. 6B shows the swing-angle detecting part 56 when the swing detecting part 56 is located as shown in FIG. 6A and moves in the counterclockwise direction and the swing-angle controlling part 13 counts 6 detection signals in total. Additionally, FIG. 6C shows the swing-angle detecting part 56 when the swing detecting part 56 is located as shown in FIG. 6A and moves in the clockwise direction and the swing-angle controlling part 13 counts 6 detection signals in total.

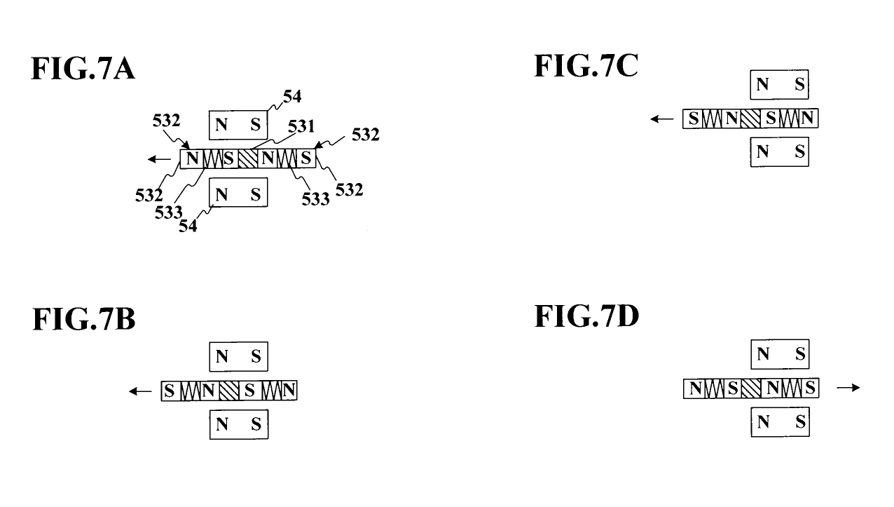

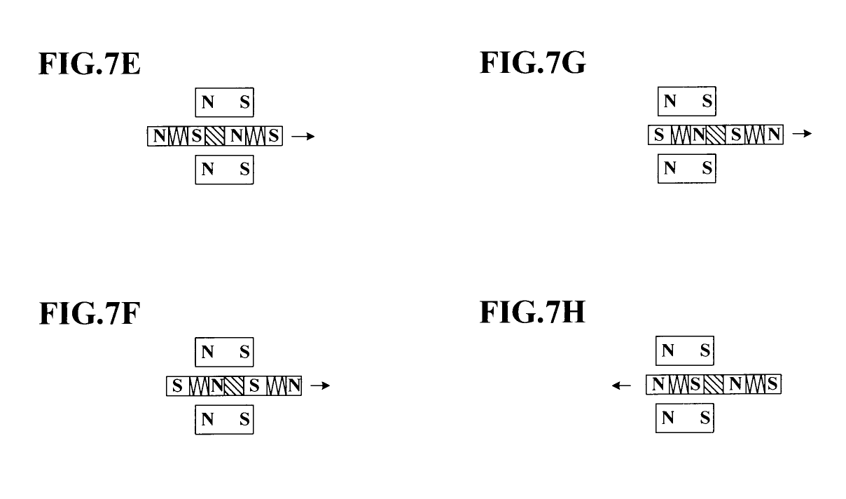

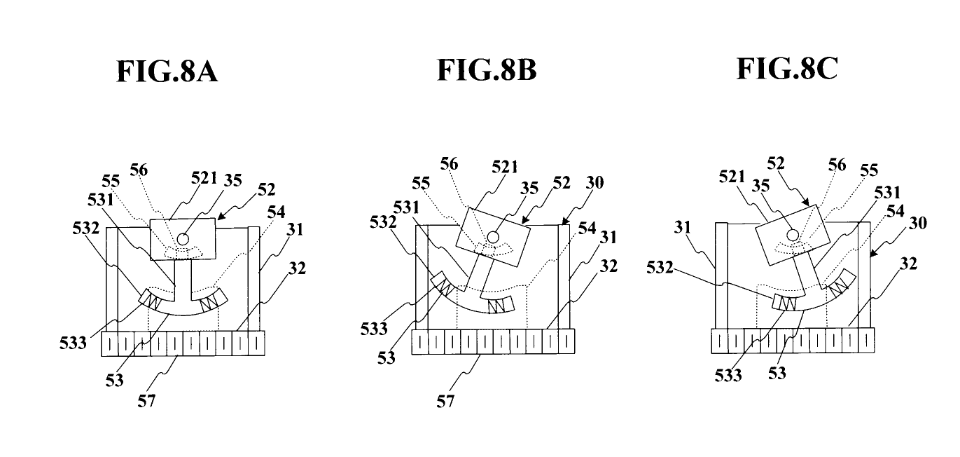

Next, the operations of the swing mechanism will be described with reference to FIG. 7A-H and FIG. 8A-C. FIG. 7A-H show a permanent magnet and an electromagnet that is swung to each position by being supplied with an alternating current, and FIG. 8A-C shows a piezoelectric vibrator that is held in a rotating member and is swung to each position. The positions of the swing-angle detecting part 56 shown in FIG. 8A, B and C correspond to the positions of the swing-angle detecting part 56 shown in FIG. 6A, B, and C, respectively.

In FIG. 7A, the S-pole of the left coil 533 is attracted to the N-pole of the permanent magnet 54, and additionally, the N-pole of the right coil 533 is attracted to the S-pole of the permanent magnet 54, thus causing the electromagnet 53 to be moved to the left.

Thereafter, the polarity of each of the left and right coils 533 is changed. The N-pole of the left coil 533 is repelled from the N-pole of the permanent magnet 54, and additionally, the S-pole of the right coil 533 is repelled from the S-pole of the permanent magnet 54. Thus, the electromagnet 53 is moved further to the left. FIG. 7B shows the electromagnet 53 when it has been moved to the left.

Thereafter, the S-pole of the right coil 533 is attracted to the N-pole of the permanent magnet 54, and additionally, the N-pole of the right coil 533 is attracted to the S-pole of the permanent magnet 54.

Thus, the electromagnet 53 is moved further to the left. FIG. 7C shows the electromagnet 53 when it has been moved further to the left.

Thereafter, the polarities of the left and right coils 533 are changed. The N-pole of the right coil 533 is repelled from the N-pole of the permanent magnet 54, and additionally, the S-pole of the right coil 533 is repelled from the S-pole of the permanent magnet 54. Thus, the moving direction of the electromagnet 53 is switched from the left to the right. FIG. 7D shows the electromagnet 53 when the moving direction thereof is switched to the right. It is noted that FIG. 8B shows the ultrasonic-wave transmitting/receiving surface 521 when it has been swung to the right in the direction opposite to the electromagnet 53 when the electromagnet 53 is moved to the left positions shown in FIGS. 7C and D.

Thereafter, the S-pole of the left coil 533 is attracted to the N-pole of the permanent magnet 54, and additionally, the N-pole of the right coil 533 is attracted to the S-pole of the permanent magnet 54.

Thus, the electromagnet 53 is moved to the right. FIG. 7E shows the electromagnet 53 when it has been moved to the right.

Thereafter, the polarity of each of left and right coils 533 is changed. The N-pole of the left coil 533 is repelled from the N-pole of the permanent magnet 54, and additionally, the S-pole of the right coil 533 is repelled from the S-pole of the permanent magnet 54. Thus, the electromagnet 53 is moved further to the right. FIG. 7F shows the electromagnet 53 when it has been moved further to the right.

Thereafter, the S-pole of the left coil 533 is attracted to the N-pole of the permanent magnet 54, and additionally, the N-pole of the left coil 533 is attracted to the S-pole of the permanent magnet 54.

Thus, the electromagnet 53 is moved further to the right. FIG. 7G shows the electromagnet 53 when it has been moved further to the right.

Thereafter, the polarity of each of the left and right coils 533 is changed. The N-pole of the left coil 533 is repelled from the N-pole of the permanent magnet 54, and additionally, the S-pole of the left coil 533 is repelled from the S-pole of the permanent magnet 54. Thus, the moving direction of the electromagnet 53 is switched from the right to the left. FIG. 7H shows the electromagnet 53 when the moving direction thereof is switched from the right to the left. It is noted that FIG. 8C shows the ultrasonic-wave transmitting/receiving surface 521 when it has been swung to the left in the direction opposite to the electromagnet 53 when the electromagnet 53 is moved to the right positions shown in FIGS. 7G and H.

Thereafter, the S-pole of the left coil 533 is attracted to the N-pole of the permanent magnet 54, and additionally, the N-pole of the right coil 533 is attracted to the S-pole of the permanent magnet 54.

Thus, the electromagnet 53 is returned to the state shown in FIG. 7A, in which the electromagnet 53 is moved to the left.

As described above, the polarity of the electromagnet 53 is changed upon receiving the voltage for driving the electromagnet, and the electromagnet 53 repeats movements alternating between the right and left directions when the coil 533 is attracted to and repelled from the permanent magnet 54. Thus, the ultrasonic-wave transmitting/receiving surface 521 of the piezoelectric vibrator 52 repeats the swing operations. It is noted that, when the electromagnet 53 is moved to the left, the ultrasonic-wave transmitting/receiving surface 521 is swung to the right. Furthermore, when the electromagnet 53 is moved to the right, the ultrasonic-wave transmitting/receiving surface 521 is swung to the left.

In addition, as described above, the timing by which the polarity of the electromagnet 53 is changed corresponds to the swing operation of the piezoelectric vibrator 52.

It is noted that, in this embodiment, the number of detection signals counted by the swing-angle controlling part 13 corresponds to the swing angle of the piezoelectric vibrator 52. For example, FIG. 6A-C show an embodiment in which, when the swing-angle controlling part 13 counts 6 detection signals in total, the direction of the current supplied with each of 2 electromagnets 53 (left and right coils 533) is switched and the direction of swinging of the piezoelectric vibrator 52 is switched. However, the present invention is not limited to this, and the swinging of the piezoelectric vibrator 52 may be made larger by increasing the number of the detection signals counted by the swing-angle controlling part 13 to 7 or more, or the swinging of the piezoelectric vibrator 52 may be made smaller by decreasing the number of the detection signals counted by the swing-angle controlling part 13 to 5 or less.

Furthermore, although in said embodiment, the magnetic body 55 is provided in the rotating member 30 and the swing-angle detecting part 56 is provided in the piezoelectric vibrator 52, the swing-angle detecting part 56 may be provided in the rotating member 30 and the magnetic body 55 may be provided in the piezoelectric vibrator 52.

Moreover, the swing-angle detecting part 56 is not limited to detecting the magnetic bodies 55 and may be configured to, for example, detect a plurality of light-emitting elements arranged in the rotating member 30.

Furthermore, although the present invention has been applied to a transesophageal probe in the abovementioned embodiment, it may be applied to a surface probe or other intraluminal probes.

It is noted that, in said embodiment, the tube 20 has been provided with separate hollow portions 21 and 22, but the tube 20 may be provided with one hollow portion so as to selectively use the hollow portions for the cable 16 and the driving cable 44.

Furthermore, although in the abovementioned embodiment, the driving cable 44 has been provided to transmit a rotating force to the rotating member 30, a feeding cable may be provided to supply electric power to the drive part for rotating the rotating member 30. The feeding cable is electrically connected to the drive part and the power source 11. Moreover, the drive part is, for example, an ultrasonic motor and is held in the recess 24 and arranged in a space to which said input gear 57 is attached. The drive axis of the ultrasonic motor is directly connected to the central axis of the rotating member 30 or indirectly connected to the central axis of the rotating member 30 via a speed reducing mechanism. Furthermore, the feeding cable may be provided separately from the cable 16 or may be included in the cable 16 together with said power supply cable.

What is claimed is:

1. An ultrasound probe comprising:
a tube having a hollow portion that is formed in a long-axis shape and is extended in the direction of said long axis as well as, at a tip end thereof, a recess with an open window allowing ultrasonic waves to pass;
a rotating member that is held in said recess and is rotated about a central axis oriented in the direction from the inside of said recess to said window;
a driving cable that passes through said hollow portion and is used for transmitting a rotating force to said rotating member, or a feeding cable that supplies an electric power to a drive part for rotating said rotating member;
a piezoelectric vibrator that has an ultrasonic-wave transmitting/receiving surface and is supported by said rotating member so as to swing said ultrasonic-wave transmitting/receiving surface about a swing axis that is orthogonal to said central axis;
a permanent magnet that is provided on one of said rotating member and said piezoelectric vibrator;
an electromagnet that is provided on the other of said rotating member and said piezoelectric vibrator so as to be opposed to said permanent magnet; and
a power supply cable that passes through said hollow portion and supplies, to said electromagnet, a current for swinging said piezoelectric vibrator.

2. The ultrasound probe according to claim 1, wherein:
said rotating member has a cylindrical shape with a cylindrical wall and a bottom and is shaped substantially coaxially with said recess;
said piezoelectric vibrator is held within said rotating member;
said swing axis is installed between opposing parts of said cylindrical wall;
said ultrasonic-wave transmitting/receiving surface is formed toward said window relative to said swing axis;
one of said permanent magnet and said electromagnet is provided toward said bottom relative to said swing axis; and
the other of said permanent magnet and said electromagnet is provided on both sides of said one magnet in said bottom.

3. The ultrasound probe according to claim 1, wherein:
said rotating member is cylindrically shaped;
an input gear is integrally provided at the bottom of said rotating member;
a rotating axis of a motor is connected to the base end of said driving cable;
a worm gear is connected to the tip end of said driving cable; and
said worm gear is engaged with said input gear via a speed reducing gear.

4. The ultrasound probe according to claim 1, comprising:
a swing-angle detecting part that detects the swing angle of said piezoelectric vibrator relative to said rotating member; and
a swing-angle controlling part that controls said current supplied to said electromagnet upon receiving a detection signal from said swing-angle detecting part.

5. The ultrasound probe according to claim 4, wherein:
a magnetic body is provided to one of said rotating member and said piezoelectric vibrator; and
said swing-angle detecting part is provided to the other of said rotating member and said piezoelectric vibrator and detects the magnetic force of said magnetic body.

6. An ultrasonic diagnosis apparatus with an ultrasound probe, the ultrasound probe comprising:
a tube having a hollow portion that is formed in a long-axis shape and is extended in the direction of said long axis as well as, at a tip end thereof, a recess with an open window allowing ultrasonic waves to pass;
a rotating member that is held in said recess and is rotated about a central axis oriented in the direction from the inside of said recess to said window;
a driving cable that passes through said hollow portion and is used for transmitting a rotating force to said rotating member, or a feeding cable that supplies an electric power to a drive part for rotating said rotating member;
a piezoelectric vibrator that has an ultrasonic-wave transmitting/receiving surface and is supported by said rotating member so as to swing said ultrasonic-wave transmitting/receiving surface about a swing axis that is orthogonal to said central axis;
a permanent magnet that is provided on one of said rotating member and said piezoelectric vibrator;
an electromagnet that is provided on the other of said rotating member and said piezoelectric vibrator so as to be opposed to said permanent magnet; and
a power supply cable that passes through said hollow portion and supplies, to said electromagnet, a current for swinging said piezoelectric vibrator.

7. The ultrasonic diagnosis apparatus according to claim 6, wherein:
said rotating member has a cylindrical shape with a cylindrical wall and a bottom and is shaped substantially coaxially with said recess;
said piezoelectric vibrator is held within said rotating member;
said swing axis is installed between opposing parts of said cylindrical wall;
said ultrasonic-wave transmitting/receiving surface is formed toward said window relative to said swing axis;
one of said permanent magnet and said electromagnet is provided toward said bottom relative to said swing axis; and
the other of said permanent magnet and said electromagnet is provided on both sides of said one magnet in said bottom.

8. The ultrasound probe according to claim 6, wherein:
said rotating member is cylindrically shaped;
an input gear is integrally provided at the bottom of said rotating member;
a rotating axis of a motor is connected to the base end of said driving cable;
a worm gear is connected to the tip end of said driving cable; and
said worm gear is engaged with said input gear via a speed reducing gear.

9. The ultrasound probe according to claim 6, comprising:
a swing-angle detecting part that detects the swing angle of said piezoelectric vibrator relative to said rotating member; and
a swing-angle controlling part that controls said current supplied to said electromagnet upon receiving a detection signal from said swing-angle detecting part.

10. The ultrasound probe according to claim 9, wherein:
a magnetic body is provided to one of said rotating member and said piezoelectric vibrator; and
said swing-angle detecting part is provided to the other of said rotating member and said piezoelectric vibrator and detects the magnetic force of said magnetic body.
